(19) United States
(12) Patent Application Publication
(10) Pub. No.: US 2023/0000786 A1
(43) Pub. Date: Jan. 5, 2023

(54) TREATMENT OF SKIN DISORDERS WITH TOPICAL COMPOSITIONS COMPRISING TAPINAROF AND A PDE4 INHIBITOR

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Karine NEIMANN, Ness Ziona (IL)

(73) Assignee: Sol-Gel Technologies Ltd., Ness Ziona (IL)

(21) Appl. No.: 17/779,151

(22) PCT Filed: Nov. 24, 2020
(86) PCT No.: PCT/IL2020/051209

§ 371 Date: May 24, 2022

Related U.S. Application Data
(60) Provisional application No. 62/967,698, filed on Jan. 30, 2020.
(60) Provisional application No. 62/939,642, filed on Nov. 24, 2019.

(51) Int. Cl. A61K 31/047; A61K 31/44; A61K 31/4035; A61K 31/69; A61K 31/203; A61K 45/06; A61K 9/00

(52) U.S. Cl. CPC A61K 31/047; A61K 31/44; A61K 31/4035; A61K 31/69; A61K 31/203; A61K 45/06; A61K 9/0014

(57) ABSTRACT

Provided herein is a topical combination composition comprising tapinarof, a PDE4 inhibitor and optionally at least one additional active agent selected from a retinoid, benzoyl peroxide (BPO), a Janus kinase inhibitor (JAK inhibitor), a corticosteroid of potency class 1-4, an acaricide and combinations thereof. The active agents in the composition of this invention are in encapsulated or non-encapsulated form, according to need. The above compositions are useful for the treatment, prevention or alleviation of a skin disorder selected from psoriasis, dermatitis, acne, rosacea, ichthyosis, scaling skin, imbalance of skin barrier and tinea and exhibit synergistic and/or additive effects allowing to reduce the active agents amounts in the combination compositions.

### FIELD OF THE INVENTION

The present disclosure, in some embodiments, relates to a treatment of a skin disorder by topical administration to a subject in need thereof a combination composition comprising tapinarof and a PDE4 inhibitor. The compositions of this invention are useful for the treatment, prevention or amelioration of skin disorders.

### BACKGROUND

Skin disorders (skin conditions) vary greatly in symptoms and in severity. They are commonly treated with systemic and/or topical medicaments. Topical medicaments, while not always available, have the advantage of avoiding systemic side-effects. On the other hand, also topical skin disorder treatments are sometimes accompanied by undesirable side-effects, especially at high doses.

The present disclosure provides novel tapinarof/PDE4 combination compositions and takes aim at minimizing undesirable side-effects.

### SUMMARY OF THE INVENTION

This invention provides a topical combination composition comprising tapinarof, a PDE4 inhibitor and optionally at least one additional active agent selected from a retinoid, benzoyl peroxide (BPO), a Janus kinase inhibitor (JAK inhibitor), a corticosteroid of potency class 1-4, an acaricide and combinations thereof. The active agents in the composition of this invention are in encapsulated or non-encapsulated form, according to need.

The above compositions are useful for the treatment, prevention or alleviation of a skin disorder selected from psoriasis, dermatitis, acne, rosacea, ichthyosis, scaling skin, imbalance of skin barrier and tinea and exhibit synergistic and/or additive effects allowing to reduce the active agents amounts in the combination compositions.

The addition of the PDE4 to tapinarof potentiates the tapinarof therapeutic effect.

### DETAILED DESCRIPTION OF THE INVENTION

The present invention provides novel topical combination compositions comprising tapinarof and a PDE4 inhibitor and methods of treatment useful for the treatment, prevention and amelioration of a skin disorder selected from psoriasis, dermatitis, acne, rosacea, ichthyosis, scaling skin, imbalance of skin barrier and tinea.

Tapinarof topical treatment is accompanied by folliculitis as a side-effect (especially at high concentrations). The compositions of the instant disclosure and the combination of tapinarof with a PDE4 inhibitor are cancelling or diminishing this side-effect. The PDE4 inhibitor in the composition of this disclosure is selected from roflumilast, apremilast, crisaborole, rolipram, cilomilast, ibudilast, piclamilast and combinations thereof.

In another embodiment, the roflumilast is roflumilast free base, roflumilast N-oxide, or pharmaceutically acceptable salt thereof.

Also provided is a topical combination composition further comprising at least one additional active agent selected from about 0.025% w/w to about 0.5% w/w of at least one retinoid; from about 2% w/w to about 10% w/w benzoyl peroxide (BPO); from about 0.1% w/w to about 3.0% w/w at least one Janus kinase inhibitor (JAK inhibitor); from about 0.01% w/w to about 0.25% w/w at least one corticosteroid of potency class 1-4; from about 0.5% to about 5% w/w at least one acaricide selected from permethrin, ivermectin and crotamiton; and combinations thereof.

It occurred to the present inventors, that topical combination compositions comprising tapinarof and a PDE4 inhibitor, optionally further comprising at least one additional active agent, may allow treatment of skin disorders for longer periods of time, exhibit improved therapeutic effects and also exhibit synergistic or additive effects in the treatment of a skin disorder. As a result, the combination compositions allow using lower dosage of the actives and diminish the product's side-effects like folliculitis, local irritation and contact dermatitis. The optional addition of at least one additional active agent selected from at least one retinoid; benzoyl peroxide (BPO); at least one Janus kinase inhibitor (JAK inhibitor); at least one corticosteroid of potency class 1-4; at least one acaricide selected from permethrin, ivermectin and crotamiton; and combinations thereof further broadens the therapeutic activity scope of the tapinarof-PDE4 active agent combination composition.

Without wishing to be bound by theory, the combination of tapinarof with a PDE4 inhibitor and optionally with an additional active agent provides additive and/or synergistic effects, enabling lower concentrations and dosages of the active agents, thus minimizing their side-effects. Both tapinarof and the PDE4 inhibitor have anti-inflammatory and anti-oxidant activities and their combinations have better activity than each of its constituents.

“Phosphodiesterase-4 (PDE4) is the major enzyme class responsible for the hydrolysis of cyclic adenosine monophosphate (cAMP), an intracellular second messenger that controls a network of proinflammatory and antiinflammatory mediators” (Textbook of Pediatric Rheumatology (Seventh Edition), 2016, Elsevier).

In some embodiments, there is provided a topical composition comprising from about 0.25% w/w to about 3.0% w/w tapinarof, from about 0.25% w/w to about 3% w/w of a PDE4 inhibitor and a carrier suitable for topical administration.

In some other embodiments, there is provided a topical combination composition further comprising about 0.025% w/w to about 0.5% w/w of at least one retinoid; from about 2% w/w to about 10% w/w benzoyl peroxide (BPO); from about 0.1% w/w to about 3.0% w/w at least one Janus kinase inhibitor (JAK inhibitor); from about 0.01% w/w to about 0.25% w/w at least one corticosteroid of potency class 1-4; from about 0.5% to about 5% w/w at least one acaricide selected from permethrin, ivermectin and crotamiton; and combinations thereof.

The tapinarof-PDE4 inhibitor combination composition of this invention has a double advantage vs. the use of tapinarof or an PDE4 inhibitor as single drugs: on the one hand the PDE4 inhibitor has the potential to alleviate the tapinarof side-effects, especially at high concentrations, and on the other hand the synergistic and/or additive effect may enable using lower active agent amounts. The addition of an PDE4 inhibitor to tapinarof potentiates the tapinarof therapeutic effect.

There is an unmet need for methods of treatment of a skin disorder using topical combination compositions comprising tapinarof, PDE4 inhibitor, and optionally additional active agent in lower doses, devoid of serious side-effects, which may be used for extended periods of time.

Active Agents in the Compositions of this Disclosure:

### Tapinarof

Tapinarof (3,5-dihydroxy-4-isopropyl-trans-stilbene, benvitimod, GSK2894512) is a first-in-class drug, whose mechanism is not yet fully understood. It is being developed by Glaxo Smith Kline (Stiefel, a GSK company) and Dermavant as a topical drug for treatment of mild to moderate psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October; 137[10]:2110-9).

According to Jancin B. (MDedge Dermatology, Nov. 11, 2017), in the above studies, the 1% tapinarof arm showed higher efficacy and had a quicker onset of action than the 0.5% arm or vehicle.

Tapinarof, which seems to be a significant advance in psoriasis treatment, presents however higher adverse effects (44.5%) when compared to placebo (20.2%) and calcipotriene (19.5%) as a single drug (Medscape Mar. 5, 2017 report by Frellick M., on Abstr. 5629, Amer. Acad. of Dermatology meeting, Mar. 4, 2017).

The most common tapinarof side-effects (>5%) across all groups treated included folliculitis (9%) and contact dermatitis (AdisInsight “Tapinarof-Dermavant Sciences/Welichem Biotech”, updated 29 Aug. 2019).

There is an unmet need for methods for the treatment of skin disorders using tapinarof topical compositions, devoid of serious side-effects.

The present invention solves the aforementioned side-effects, inter alia by encapsulating tapinarof by a process detailed in Examples 1 and 2 (see also U.S. Pat. No. 9,687,465 and published U.S. Patent Application No. 2018147165 (to Sol-Gel Technologies)).

The tapinarof encapsulation process detailed in Examples 1 and 2 allows the use of tapinarof concentrations equal or higher than 1% w/w with minimal or no side-effects.

In addition, the encapsulation process detailed in Examples 1 and 2 improves the chemical stability of tapinarof in multi-component combination compositions.

The additive or synergistic effects of tapinarof with at least one PDE4 inhibitor active agent and optionally at least one additional active agent allow reducing the amounts of the active agents (including the amount of tapinarof) in the composition of this invention.

Without Wishing to be Bound by Theory, Combining Tapinarof with a PDE4 Inhibitor cancels or diminishes the tapinarof folliculitis side-effect.

### PDE4 Inhibitor (PDE4I)

Phosphodiesterase Type 4 inhibitors are blocking the degradation of cyclic adenosine monophosphate (cAMP) by phosphodiesterase 4 (PDE4).

PDE4 inhibitors have been investigated for a number of medical indications, like CNS disorders, chronic obstructive pulmonary disease (COPD), asthma and rheumatoid arthritis.

The systemic administration of PDE4 inhibitors is accompanied by emesis, which is a big drawback. The topical administration of PDE4 inhibitor comprising combination drugs provided in this disclosure avoids said systemic emetic effect.

The PDE4 inhibitor in the composition of this disclosure is selected from roflumilast, roflumilast N-oxide, apremilast, crisaborole, rolipram, cilomilast, ibudilast, piclamilast, pharmaceutically acceptable salt thereof and combinations thereof.

In another embodiment, the roflumilast is roflumilast free base, roflumilast N-oxide, or pharmaceutically acceptable salt thereof.

### Retinoid

Retinoids are a class of chemical compounds structurally related to Vitamin A which are effective in the treatment of a number of skin disorders, like acne and psoriasis.

Long-term high intake of retinoids causes a number of side-effects.

The retinoids belong to several generations and the main members of this group are tretinoin, adapalene and tazarotene.

Tretinoin, a first-generation retinoid (also known as all-trans retinoic acid or ATRA) is used topically for the treatment of acne.

Adapalene, a third-generation retinoid, is used topically for the treatment of mild to moderate acne.

Tazarotene (marketed as Tazorac, Avage, Zorac and Fabior) is a third-generation prescription topical retinoid sold as a cream, gel, or foam. Tazarotene exhibits side-effects like itching, redness, burning and stinging, especially on long-term treatment.

Treatment for extended periods of time is important, for example for the therapy of a chronic skin disorder, and the composition of this invention allows long-term treatment.

### Benzoyl Peroxide (BPO)

BPO topical gel (alone or in combination with another active agent selected from adapalene, clindamycin, erythromycin) is used the topical treatment of acne.

Topical BPO treatment is accompanied by side-effects like skin irritation, itching, peeling and reddened skin. The encapsulated-BPO compositions of the instant disclosure reduce these BPO side-effects.

Due to its peroxide chemical structure, BPO presents several problems:
a. BPO is a strong oxidant, which may compromise the chemical stability of the other active agents in the combination compositions of this invention; and
b. Side-effects like skin irritation, itching, peeling and reddened skin.

The BPO-comprising compositions of this invention use micronized BPO as raw-material and several solutions to the above side-effects:
(i) BPO encapsulation according to Examples 1 and 2, U.S. Pat. No. 9,687,465 and published U.S. Patent Application No. 2018147165 (to Sol-Gel Technologies), whose contents are enclosed herein in their entirety, thus protecting the other active agents in a BPO-combination composition from the BPO oxidation effect and minimizing the BPO skin irritation side-effect.
(ii) Topical application of the BPO-comprising compositions of this invention to the affected skin area of a subject in need thereof as two separate compositions (simultaneously or sequentially in either order) to be mixed on the subject's skin, the first composition comprising tapinarof, a PDE4 inhibitor and a carrier suitable for topical administration and the second composition comprising benzoyl peroxide and a carrier suitable for topical administration (see Example 7). Due to this mode of administration, BPO does not compromise the chemical stability of the other active agents in the combination compositions of this disclosure. The administration can be done for example by applying the two separate compositions to the affected area of the skin of a subject in need thereof from two application syringes or from a dual chamber application syringe, simultaneously or sequentially in either order. In a preferred product according to the invention, the first and second compositions are respectively filled in the suitable ratio in the two chambers of a dual chamber dispensing system of the type described in EP-A-0644129 and U. S. Pat. No. 5, 356,040, the contents of which are incorporated herein by reference. Such a system has two side-by-side chambers, each equipped with a dispensing valve; these are operated by adjacent actuators so as to dispense the formulations either simultaneously or separately as desired. Suitable dispensing systems having chambers which are each capable of holding about 15 ml of composition, are available from Maplast S. r. 1., Via Pasublo 3, Tradate 21049 VA, Italy. The respective dimensions of the dispenser means may be chosen to provide dispensing of the respective compositions in a predetermined ratio (see Example 7).

### Janus Kinase Inhibitors (JAK Inhibitors)

JAK inhibitors are a class of drugs interfering with the JAK-STAT signaling pathway by inhibiting at least one of the Janus kinase enzymes JAK1, JAK2, JAK3 or TYK2. Some JAK inhibitors inhibit all the above enzymes and are therefore named pan-JAK inhibitors.

The at least one JAK inhibitor in the compositions of this invention is selected from a JAK1 inhibitor, a JAK2 inhibitor, a JAK3 inhibitor, a TYK2 inhibitor a pan-JAK inhibitor and combinations thereof.

The preferred at least one JAK inhibitor in the compositions of this invention is selected from tofacitinib, abrocitinib, delgocitinib, ruxolitinib and combinations thereof.

### Corticosteroids

The tapinarof-PDE4 combination composition of this invention may further comprise an additional active agent such as from about 0.01% w/w to about 0.25% w/w at least one corticosteroid of potency class 1-4, approved for topical use.

The optional corticosteroid in the compositions may be superpotent (Class 1) or potent (Class 2). Alternatively, the steroid may be of lower potency (Class 3-4), thus minimizing the steroid side-effects, such as the risk of pituitary suppression.

According to the “Topical steroid potency chart” of the National Psoriasis Foundation (NPF), the various marketed topical drugs comprising steroids belong to the following potency classes, according to the steroid and the topical drug strength. Due to the different topical drug strength, drugs of different strengths and/or different dosage forms may belong to more than one steroid class.

The percentages in parentheses are the steroid strengths for the FDA-approved topical steroid compositions of potency classes 1-4.

Class 1—superpotent, comprising 7 steroids: clobetasol propionate (0.05%), flurandrenolide (0.05%), betamethasone dipropionate (0.05%), diflorasone diacetate (0.05%), desoxymethasone or fluocinonide (0.1%).

Class 2—potent, comprising 6 steroids: betamethasone dipropionate (0.05%), mometasone furoate (0.1%), diflorasone diacetate (0.05%), halcinonide (0.1%), fluocinonide (0.05%) or desoxymethasone (0.05%-0.25%).

Class 3—upper mid-strength, comprising 3 steroids: fluticasone propionate (0.005%), fluocinonide (0.05%) or betamethasone valerate (0.12%).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) or hydrocortisone valerate (0.2%).

In some embodiments, there is provided a topical composition comprising from about 0.25% w/w to about 3.0% w/w tapinarof, from about 0.25% w/w to about 3% w/w at least one PDE4 inhibitor, from about 0.01% w/w to about 0.25% w/w at least one corticosteroid of potency class 1-4 and a carrier suitable for topical administration.

### Acaricides

Some of the skin disorders treated with the compositions of this invention are caused i.a. by parasites like ticks and mites. For example, rosacea is caused i.a. by Demodex folliculorum.

The acaricide activity of tapinarof in the tapinarof-PDE4 combination composition is augmented by addition of an acaricide due to an additive or synergistic effect, allowing to reduce the amounts of the active agents in the composition.

The preferred at least one acaricide in the compositions of this invention is selected from permethrin, ivermectin, crotamiton and combinations thereof.

Skin Disorders Treated with the Compositions of this Disclosure

The skin disorders treated with the compositions of this disclosure are selected from psoriasis (selected from plaque psoriasis, flexural/inverse psoriasis, scalp psoriasis, sebopsoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis; scaling skin; imbalance of skin barrier; and tinea selected from tinea pedis, tinea capitis, tinea barbae, tinea faciei, tinea corporis, tinea manum, tinea cruris and tinea interdigital.

### Acne

Acne is a chronic inflammatory disease of the pilosebaceous unit resulting from androgen-induced increased sebum production, altered keratinization, inflammation, and bacterial colonisation of hair follicles on the face, neck, chest, and back by Propionibacterium acnes (P. acnes). Although early colonisation with P. acnes and family history might have important roles in the disease, exactly what triggers acne and how treatment affects the course of the disease remains unclear (Williams H. C. et al., The Lancet, Vol. 379. January 2012, pp. 361-372).

There is no ideal treatment for acne. Good quality evidence on comparative effectiveness of common topical and systemic acne therapies is scarce. Topical therapies including benzoyl peroxide, retinoids, and antibiotics when used in combination usually improve control of mild to moderate acne, but suffer from side-effects. Treatment with combined oral contraceptives can help women with acne. Patients with more severe inflammatory acne usually need oral antibiotics combined with topical benzoyl peroxide to decrease antibiotic-resistant organisms.

Oral isotretinoin is the most effective therapy and is used early in severe disease, but its use is limited by teratogenicity and other side-effects.

The treatment of acne with the composition of this invention allows using lower amounts of active agents and therefore results in reduced side-effects, due to the additive and/or synergistic effects.

### Rosacea (Acne Rosacea)

Rosacea is a chronic skin disease that affects more than 16 million Americans. The cause of rosacea is still unknown, and there is no cure. However, research has allowed doctors to find ways to treat the condition by minimizing its symptoms (Cynthia Cobb, Healthline May 21, 2018).

There are four subtypes of rosacea. Each subtype has its own set of symptoms. It is possible to have more than one subtype of rosacea at a time.

Rosacea's typical symptom is small, red, pus-filled bumps on the skin that are present during flare-ups. Typically, rosacea affects only skin on the nose, cheeks, and forehead.

Flare-ups often occur in cycles. This means that one will experience symptoms for weeks or months at a time, the symptoms will go away, and then return.

The four types of rosacea are:

Subtype one, known as erythematotelangiectatic rosacea (ETR), is associated with facial redness, flushing, and visible blood vessels.

Subtype two, papulopustular (or acne) rosacea, is associated with acne-like breakouts, and often affects middle-aged women.

Subtype three, known as rhinophyma, is a rare form associated with thickening of the skin on your nose. It usually affects men and is often accompanied by another subtype of rosacea.

Subtype four is known as ocular rosacea, and its symptoms are centered on the eye area.

### Psoriasis

Psoriasis is an autoimmune disease, characterized by typically red, scaly patches of skin. There are five main types of psoriasis: plaque, guttate, flexural/inverse, pustular, and erythrodermic. Psoriasis vulgaris is the most common form of psoriasis.

Though a number of psoriasis treatments are available, most treatments bring about symptom alleviation or remission rather than complete cure.

### Imbalance of Skin Barrier

The skin barrier imbalance is an important element in many skin disorders. A disrupted skin barrier allows entry of allergens and lead to systemic allergic responses.

The epidermal protein filaggrin plays an important role in skin barrier.

The instant disclosure provides tapinarof-PDE4 combinations which restore the skin barrier balance, due to the synergistic effect of tapinarof and PDE4, acting in tandem by different mechanisms of action.

### Topical Tapinarof Combination Compositions

Provided herein are compositions, combinations, kits and articles of manufacture that include tapinarof in combination with a PDE4 inhibitor and optionally at least one additional active agent, for treating a skin disorder selected from psoriasis, dermatitis, acne, rosacea, ichthyosis, scaling skin, imbalance of skin barrier and tinea.

The compositions, combinations and articles of manufacture can be administered using a variety of routes such as topical application or transdermal application. The preferred route is the topical route and the preferred formulations are the cream, the gel and the lotion.

Therapeutically effective amounts of tapinarof, PDE4 inhibitor and optionally an additional active agent are mixed with a suitable pharmaceutical carrier or vehicle suitable for topical use, for the treatment, prevention or alleviation of the symptoms manifested by a skin disorder.

Tapinarof, at least one PDE4 inhibitor and optionally and at least one additional active agent in the combination compositions are included in an amount effective for treating, preventing or alleviating the skin disorder symptoms. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof, a PDE4 inhibitor and optionally an additional active agent in the marketed single drug currently administered for the treatment of a skin disorder. The dosage and regimen of administration may be determined by dose finding studies, as known in the art.

Exemplary dosages, strengths and concentrations of tapinarof in the tapinarof-PDE4 inhibitor or tapinarof-PDE4 inhibitor-additional active agent combination compositions administered topically, can be in the range of from about or at 0.1%, 0.25%, 0.5%, 1%, 2% or 3% w/w. Typical strengths of tapinarof in the topical combination compositions of this invention are 0.25%, 0.5% or 1%, 2% and 3% w/w.

TABLE 1
Exemplary Combinations of Active Agents Classes

| Act. Agent | | | | | | | |
|---|---|---|---|---|---|---|---|
| Class/Comb. | TAP | PDE4i | RET | BPO | JAKi | CORT | AC |
| TAP/PDE4 | + | + | | | | | |
| TAP/PDE4i/RET | + | + | + | | | | |
| TAP/PDE4i/BPO | + | + | | + | | | |
| TAP/PDE4i/JAKi | + | + | | | + | | |
| TAP/PDE4i/CORT | + | + | | | | + | |
| TAP/PDE4i/AC | + | + | | | | | + |

Legend: Tapinarof (TAP), PDE4 inhibitor (PDE4i), Retinoid (RET), Benzoyl peroxide (BPO), JAK Inhibitor (JAKi), Acaricide (AC)

TABLE 2
Exemplary Combinations of Specific Active Agents

| Act. Agent | | | | | | | |
|---|---|---|---|---|---|---|---|
| Class/Comb. | TAP | PDE4i | RET | BPO | JAKi | CORT | AC |
| TAP/ROFL | + | + | | | | | |
| TAP/APRE | + | + | | | | | |
| TAP/ROFL/TRET | + | + | + | | | | |
| TAP/ROFL/ADA | + | + | + | | | | |
| TAP/ROFL/TAZ | + | + | + | | | | |
| TAP/ROFL/BPO | + | + | | + | | | |
| TAP/ROFL/TOFA | + | + | | | + | | |
| TAP/ROFL/ABRO | + | + | | | + | | |
| TAP/ROFL/DELG | + | + | | | + | | |
| TAP/ROFL/RUXO | + | + | | | + | | |
| TAP/ROFL/HAL | + | + | | | | + | |
| TAP/ROFL/PER | + | + | | | | | + |
| TAP/ROFL/IVER | + | + | | | | | + |
| TAP/ROFL/CROT | + | + | | | | | + |

Legend: Tapinarof (TAP), PDE4 inhibitor (PDE4i), Retinoid (RET), Benzoyl peroxide (BPO), JAK Inhibitor (JAKi), Acaricide (AC). Roflumilast (ROFL), Apremilast (APRE), Tretinoin (TRET), Adapalene (ADA), Tazarotene (TAZ), Tofacitinib (TOFA), Abrocitinib (ABRO), Delgocitinib (DELG), Ruxolitinib (RUXO) Halobetasol (HAL), Permethrin (PER), Ivermectin (IVER), Crotamiton (CROT).

Table 3: Exemplary active agents' combination compositions 1-20:

(see also Tables 1 and 2 above and Examples 1-50):

The active agents in the combination compositions 1-20 described hereinbelow may be encapsulated (see Examples 1-7) or non-encapsulated (Examples 8-10):
1. A topical composition comprising from about 0.25% w/w to about 3.0% w/w tapinarof, from about 0.25% w/w to about 3% w/w at least one PDE4 inhibitor selected from roflumilast, apremilast, crisaborole, rolipram, cilomilast, ibudilast, piclamilast and combinations thereof and a carrier suitable for topical administration in the treatment, prevention or alleviation of a skin disorder selected from psoriasis, dermatitis, acne, rosacea, ichthyosis, scaling skin, imbalance of skin barrier and tinea.
2. A topical composition comprising from about 0.25% w/w to about 3.0% w/w tapinarof, from about 0.25% w/w to about 3% w/w roflumilast and a carrier suitable for topical administration in the treatment, prevention or alleviation of a skin disorder selected from psoriasis, dermatitis, acne, rosacea, ichthyosis, scaling skin, imbalance of skin barrier and tinea.
3. A topical composition comprising from about 0.25% w/w to about 3.0% w/w tapinarof, from about 0.25% w/w to about 3% w/w apremilast and a carrier suitable for topical administration in the treatment, prevention or alleviation of a skin disorder selected from psoriasis, dermatitis, acne, rosacea, ichthyosis, scaling skin, imbalance of skin barrier and tinea.
4. A topical composition comprising from about 0.25% w/w to about 3.0% w/w tapinarof, from about 0.25% w/w to about 3% w/w at least one PDE4 inhibitor selected from roflumilast, apremilast, crisaborole, rolipram. cilomilast, ibudilast, piclamilast and combinations thereof, from about 0.025% w/w to about 0.5% w/w of at least one retinoid selected from tretinoin, adapalene and tazarotene and a carrier suitable for topical administration in the treatment, prevention or alleviation of a skin disorder selected from psoriasis, dermatitis, acne, rosacea, ichthyosis, scaling skin, imbalance of skin barrier and tinea.
5. A topical composition comprising from about 0.25% w/w to about 3.0% w/w tapinarof, from about 0.25% w/w to about 3% w/w roflumilast, from about 0.025% w/w to about 0.5% w/w tretinoin and a carrier suitable for topical administration in the treatment, prevention or alleviation of a skin disorder selected from psoriasis, dermatitis, acne, rosacea, ichthyosis, scaling skin, imbalance of skin barrier and tinea.
6. A topical composition comprising from about 0.25% w/w to about 3.0% w/w tapinarof, from about 0.25% w/w to about 3% w/w roflumilast, from about 0.025% w/w to about 0.5% w/w adapalene and a carrier suitable for topical administration in the treatment, prevention or alleviation of a skin disorder selected from psoriasis, dermatitis, acne, rosacea, ichthyosis, scaling skin, imbalance of skin barrier and tinea.
7. A topical composition comprising from about 0.25% w/w to about 3.0% w/w tapinarof, from about 0.25% w/w to about 3% w/w apremilast, from about 0.025% w/w to about 0.5% w/w tazarotene and a carrier suitable for topical administration in the treatment, prevention or alleviation of a skin disorder selected from psoriasis, dermatitis, acne, rosacea, ichthyosis, scaling skin, imbalance of skin barrier and tinea.
8. A topical composition comprising from about 0.25% w/w to about 3.0% w/w tapinarof, from about 0.25% w/w to about 3% w/w at least one PDE4 inhibitor selected from roflumilast, apremilast, crisaborole, rolipram. cilomilast, ibudilast, piclamilast and combinations thereof, from about 2% w/w to about 10% w/w benzoyl peroxide (BPO) and a carrier suitable for topical administration in the treatment, prevention or alleviation of a skin disorder selected from psoriasis, dermatitis, acne, rosacea, ichthyosis, scaling skin, imbalance of skin barrier and tinea.
9. A topical composition comprising from about 0.25% w/w to about 3.0% w/w tapinarof, from about 0.25% w/w to about 3% w/w roflumilast, from about 2% w/w to about 10% w/w benzoyl peroxide (BPO) and a carrier suitable for topical administration in the treatment, prevention or alleviation of a skin disorder selected from psoriasis, dermatitis, acne, rosacea, ichthyosis, scaling skin, imbalance of skin barrier and tinea.
10. A topical composition comprising from about 0.25% w/w to about 3.0% w/w tapinarof, from about 0.25% w/w to about 3% w/w at least one PDE4 inhibitor selected from roflumilast, apremilast, crisaborole, rolipram. cilomilast, ibudilast, piclamilast and combinations thereof, from about 0.1% w/w to about 3.0% w/w at least one Janus kinase inhibitor (JAK inhibitor) selected from tofacitinib, abrocitinib, delgocitinib, ruxolitinib and combinations thereof and a carrier suitable for topical administration in the treatment, prevention or alleviation of a skin disorder selected from psoriasis, dermatitis, acne, rosacea, ichthyosis, scaling skin, imbalance of skin barrier and tinea.
11. A topical composition comprising from about 0.25% w/w to about 3.0% w/w tapinarof, from about 0.25% w/w to about 3% w/w roflumilast, from about 0.1% w/w to about 3.0% w/w tofacitinib and a carrier suitable for topical administration in the treatment, prevention or alleviation of a skin disorder selected from psoriasis, dermatitis, acne, rosacea, ichthyosis, scaling skin, imbalance of skin barrier and tinea.
12. A topical composition comprising from about 0.25% w/w to about 3.0% w/w tapinarof, from about 0.25% w/w to about 3% w/w roflumilast, from about 0.1% w/w to about 3.0% w/w abrocitinib and a carrier suitable for topical administration in the treatment, prevention or alleviation of a skin disorder selected from psoriasis, dermatitis, acne, rosacea, ichthyosis, scaling skin, imbalance of skin barrier and tinea.
13. A topical composition comprising from about 0.25% w/w to about 3.0% w/w tapinarof, from about 0.25% w/w to about 3% w/w roflumilast, from about 0.1% w/w to about 3.0% w/w delgocitinib and a carrier suitable for topical administration in the treatment, prevention or alleviation of a skin disorder selected from psoriasis, dermatitis, acne, rosacea, ichthyosis, scaling skin, imbalance of skin barrier and tinea.
14. A topical composition comprising from about 0.25% w/w to about 3.0% w/w tapinarof, from about 0.25% w/w to about 3% w/w roflumilast, from about 0.1% w/w to about 3.0% w/w ruxolitinib and a carrier suitable for topical administration in the treatment, prevention or alleviation of a skin disorder selected from psoriasis, dermatitis, acne, rosacea, ichthyosis, scaling skin, imbalance of skin barrier and tinea.
15. A topical composition comprising from about 0.25% w/w to about 3.0% w/w tapinarof, from about 0.25% w/w to about 3% w/w at least one PDE4 inhibitor selected from roflumilast, apremilast, crisaborole, rolipram. cilomilast, ibudilast, piclamilast and combinations thereof, from about 0.01% w/w to about 0.25% w/w at least one corticosteroid of potency class 1˜4 and a carrier suitable for topical administration in the treatment, prevention or alleviation of a skin disorder selected from selected from psoriasis, dermatitis, acne, rosacea, ichthyosis, scaling skin, imbalance of skin barrier and tinea.
16. A topical composition comprising from about 0.25% w/w to about 3.0% w/w tapinarof, from about 0.25% w/w to about 3% w/w roflumilast, from about 0.01% w/w to about 0.25% w/w halobetasol and a carrier suitable for topical administration in the treatment, prevention or alleviation of a skin disorder selected from selected from psoriasis, dermatitis, acne, rosacea, ichthyosis, scaling skin, imbalance of skin barrier and tinea.
17. A topical composition comprising from about 0.25% w/w to about 3.0% w/w tapinarof, from about 0.25% w/w to about 3% w/w at least one PDE4 inhibitor selected from roflumilast, apremilast, crisaborole, rolipram. cilomilast, ibudilast, piclamilast and combinations thereof, from about 0.5% to about 5% w/w at least one acaricide selected from permethrin, ivermectin and crotamiton and combinations thereof and a carrier suitable for topical administration in the treatment, prevention or alleviation of a skin disorder selected from psoriasis, dermatitis, acne, rosacea, ichthyosis, scaling skin, imbalance of skin barrier and tinea.
18. A topical composition comprising from about 0.25% w/w to about 3.0% w/w tapinarof, from about 0.25% w/w to about 3% w/w roflumilast, from about 0.5% to about 5% w/w permethrin and a carrier suitable for topical administration in the treatment, prevention or alleviation of a skin disorder psoriasis, dermatitis, acne, rosacea, ichthyosis, scaling skin, imbalance of skin barrier and tinea.
19. A topical composition comprising from about 0.25% w/w to about 3.0% w/w tapinarof, from about 0.25% w/w to about 3% w/w roflumilast, from about 0.5% to about 5% w/w ivermectin and a carrier suitable for topical administration in the treatment, prevention or alleviation of a skin disorder psoriasis, dermatitis, acne, rosacea, ichthyosis, scaling skin, imbalance of skin barrier and tinea.
20. A topical composition comprising from about 0.25% w/w to about 3.0% w/w tapinarof, from about 0.25% w/w to about 3% w/w roflumilast, from about 0.5% to about 5% w/w crotamiton and a carrier suitable for topical administration in the treatment, prevention or alleviation of a skin disorder. psoriasis, dermatitis, acne, rosacea, ichthyosis, scaling skin, imbalance of skin barrier and tinea.

The frequency of administration of the composition of this invention can be determined empirically. Exemplary frequencies are once daily, twice daily, weekly, bi-weekly and monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams or any other formulation suitable for topical administration. The preferred compositions are the cream, the gel and the lotion.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the skin disorder, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Methods of Treatment

According to an aspect of the invention, there is provided a method of treatment of a skin disorder selected from psoriasis, dermatitis, acne, rosacea, ichthyosis, scaling skin, imbalance of skin barrier and tinea, by treatment of a subject in need thereof with a combination composition of tapinarof-PDE4 inhibitor or tapinarof-PDE4 inhibitor-additional active agent.

The combination of tapinarof with at least one PDE4 inhibitor also cancels or diminishes the tapinarof folliculitis side-effect.

In some embodiments, the effective amount is a therapeutically effective amount of tapinarof and an PDE4 inhibitor or of tapinarof, an PDE4 inhibitor and at least one additional active agent, namely an amount which will cure, treat, alleviate or prevent a skin disorder selected from psoriasis, dermatitis, acne, rosacea, ichthyosis, scaling skin, imbalance of skin barrier and tinea.

In some embodiments, co-administration of tapinarof and a PDE4 inhibitor or of tapinarof, a PDE4 inhibitor and at least one additional active agent provides an additive and/or synergistic effect while treating, preventing or alleviating a skin disorder.

In some other embodiments, the co-administration may be made either by administration of a single combination composition, or alternatively by separate administration of a first composition comprising tapinarof and an PDE4 inhibitor and a second composition comprising at least one additional active agent.

### Regimen of Administration of the Tapinarof—Pde4 Inhibitor—Optional Additional Active Agent Topical Combination Compositions

Therapeutically effective concentrations of the active agents in the compositions of this invention for treatment, prevention or alleviation of the symptoms manifested by a skin disorder are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and an PDE4 inhibitor and optionally an additional active agent in the developed or marketed single drug currently being developed or used for the treatment of a skin disorder. The dosage and regimen of administration may be determined by dose finding studies, as known in the art.

Exemplary dosages, strengths and concentrations of tapinarof in the topical combination compositions of this invention, are in the range of from about 0.25% w/w to about 3% w/w or at 0.25%, 0.5%, 1%, 2% or 3% w/w. Typical strengths in the topical combination compositions of this invention are 0.25%, 0.5%, 1% or 3% w/w tapinarof.

Exemplary dosages, strengths and concentrations of the at least one PDE4 inhibitor in the topical combination compositions of this invention, are in the range of from about 0.25% w/w to about 3% w/w or at 0.25%, 0.5%, 1%, 2% or 3% w/w. Typical strengths in the topical combination compositions of this invention are 0.25%, 0.5%, 1% or 2% w/w at least one PDE4 inhibitor.

Exemplary strengths and concentrations of the least one JAK inhibitor in the topical combination compositions are 0.1%, 0.25%, 0.5%, 1%, 2% or 3% w/w. Typical strengths in the topical combination compositions of this invention are 0.1%, 0.25%, 0.5% or 1% w/w.

Exemplary strengths and concentrations of BPO in the topical combination compositions comprising BPO are 2%, 3%, 4%, 5%, 6%, 7%, 8%, 9% or 10% w/w. Typical strengths in the topical combination compositions of this invention are 5%, or 10% w/w.

Exemplary strengths and concentrations of the least one retinoid in the topical combination compositions are 0.025%, 0.05%, 0.1%, 0.15%, 0.2%, 0.25%, 0.3%, 0.4% or 0.5% w/w. Typical strengths in the topical combination compositions of this invention are 0.05%, or 0.1% w/w.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### Kits

Kits containing the combination compositions optionally including instructions for administration are provided. The combinations include, for example, the compositions as provided herein. Additionally, provided herein are kits containing the above-described combinations and optionally instructions for administration by topical, transdermal, or other routes, depending on the composition to be delivered.

The compositions provided herein can be packaged as articles of manufacture containing packaging material, a composition provided herein, and a label that indicates that the composition is for treating a skin disorder, and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Embodiments

In some embodiments, there is provided a topical composition comprising from about 0.25% w/w to about 3.0% w/w tapinarof, from about 0.25% w/w to about 3.0% w/w at least one PDE4 inhibitor and a carrier suitable for topical administration.

In some embodiments, there is provided a composition comprising from about 0.25% w/w to about 3.0% w/w tapinarof, from about 0.25% w/w to about 3.0% w/w at least one PDE4 inhibitor and a carrier suitable for topical administration, wherein the at least one PDE4 inhibitor is selected from roflumilast, roflumilast N-oxide, apremilast, crisaborole, rolipram, cilomilast, ibudilast, piclamilast, pharmaceutically acceptable salts thereof, and combinations thereof.

In some embodiments, there is provided a composition comprising from about 0.25% w/w to about 3.0% w/w tapinarof, from about 0.25% w/w to about 3.0% w/w at least one PDE4 inhibitor and a carrier suitable for topical administration, wherein the at least one PDE4 inhibitor is roflumilast or pharmaceutically acceptable salt thereof.

In some embodiments, there is provided a composition comprising from about 0.25% w/w to about 3.0% w/w tapinarof, from about 0.25% w/w to about 3.0% w/w at least one PDE4 inhibitor and a carrier suitable for topical administration, wherein the at least one PDE4 inhibitor is roflumilast N-oxide.

In some embodiments, there is provided a composition comprising from about 0.25% w/w to about 3.0% w/w tapinarof, from about 0.25% w/w to about 3.0% w/w at least one PDE4 inhibitor and a carrier suitable for topical administration, wherein the at least one PDE4 inhibitor is apremilast.

In some embodiments, there is provided a composition comprising from about 0.25% w/w to about 3.0% w/w tapinarof, from about 0.25% w/w to about 3.0% w/w at least one PDE4 inhibitor and a carrier suitable for topical administration, wherein the at least one PDE4 inhibitor is crisaborole.

In some embodiments, there is provided a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram, cilomilast, ibudilast, piclamilast, pharmaceutically acceptable salt thereof and combinations thereof, further comprising at least one additional active agent selected from about 0.025% w/w to about 0.5% w/w of at least one retinoid; from about 2% w/w to about 10% w/w benzoyl peroxide (BPO); from about 0.1% w/w to about 3.0% w/w at least one Janus kinase inhibitor (JAK inhibitor); from about 0.01% w/w to about 0.25% w/w at least one corticosteroid of potency class 1-4; from about 0.5% to about 5% w/w at least one acaricide; and combinations thereof and a carrier suitable for topical administration.

In some embodiments, there is provided a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram, cilomilast, ibudilast, piclamilast, pharmaceutically acceptable salt thereof and combinations thereof, further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof and a carrier suitable for topical administration, wherein said composition consisting of two separate compositions, a first composition comprising tapinarof and at least one PDE4 inhibitor and a second composition comprising at least one additional active agent selected from a retinoid, benzoyl peroxide (BPO), a Janus kinase inhibitor (JAK inhibitor), a corticosteroid of potency class 1-4, an acaricide and combinations thereof.

In some embodiments, there is provided a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram, cilomilast, ibudilast, piclamilast, pharmaceutically acceptable salt thereof and combinations thereof, further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof and a carrier suitable for topical administration, wherein said composition consisting of two separate compositions, a first composition comprising tapinarof and at least one PDE4 inhibitor and a second composition comprising at least one additional active agent selected from a retinoid, benzoyl peroxide (BPO), a Janus kinase inhibitor (JAK inhibitor), a corticosteroid of potency class 1-4, an acaricide and combinations thereof, wherein the at least one retinoid is selected from tretinoin, adapalene, tazarotene and combinations thereof, wherein the at least one JAK inhibitor is selected from tofacitinib, abrocitinib, delgocitinib, ruxolitinib and combinations thereof and wherein the at least one acaricide is selected from permethrin, ivermectin, crotamiton and combinations thereof.

In some embodiments, there is provided a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram, cilomilast, ibudilast, piclamilast, pharmaceutically acceptable salt thereof and combinations thereof, optionally further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof and a carrier suitable for topical administration, wherein at least one of the active agents selected from tapinarof, at least one PDE4 inhibitor and the optional at least one additional active agent is encapsulated.

In some embodiments, there is provided a dosage form comprising a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram, cilomilast, ibudilast, piclamilast, pharmaceutically acceptable salt thereof and combinations thereof, optionally further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof and a carrier suitable for topical administration, wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof. In some embodiments, according to the method of the present invention, the topical administration is made to the affected skin area of a subject in need thereof as two separate compositions, a first composition comprising tapinarof and said at least one PDE4 inhibitor and a second composition comprising said at least one additional active agent, to be administered from two application syringes or from a dual chamber application syringe, simultaneously or sequentially in either order, wherein the two compositions are mixed on the skin of said subject in need thereof.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is selected from psoriasis, dermatitis, acne, rosacea, ichthyosis, scaling skin, imbalance of skin barrier and tinea.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is psoriasis, selected from plaque psoriasis, scalp psoriasis, sebopsoriasis and genital psoriasis.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is acne, selected from acne vulgaris, papulopustular acne and nodular acne.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is rosacea, selected from erythematotelangiectatic rosacea, papulopustular rosacea, rhinophyma rosacea and ocular rosacea.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is dermatitis (eczema), selected from atopic dermatitis, contact dermatitis, dyshidrotic dermatitis, nummular dermatitis and seborrheic dermatitis.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is tinea, selected from tinea pedis, tinea capitis, tinea barbae, tinea faciei, tinea corporis, tinea manum, tinea cruris and tinea interdigital.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is selected from psoriasis, dermatitis, acne, rosacea, ichthyosis, scaling skin, imbalance of skin barrier and tinea and wherein the treatment comprises once daily or twice daily topical administration to a skin disorder subject in need thereof of a therapeutically effective amount of said composition.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is selected from psoriasis, dermatitis, acne, rosacea, ichthyosis, scaling skin, imbalance of skin barrier and tinea, wherein tapinarof, the at least one PDE4 inhibitor and the optional at least one additional active agent exhibit an additive or synergistic effect, thereby allowing to reduce the amounts of the active agents in the composition.

In some embodiments, there is provided a method of treatment, prevention or alleviation of a skin disorder, by topical administration to the affected area of a skin disorder subject in need thereof of a therapeutically effective amount of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the skin disorder is selected from psoriasis, dermatitis, acne, rosacea, ichthyosis, scaling skin, imbalance of skin barrier and tinea, wherein said at least one PDE4 inhibitor cancels or diminishes the tapinarof folliculitis side-effect.

In some embodiments, there is provided a regimen of administration comprising the once daily or twice daily administration to a skin disorder subject in need thereof of a therapeutically effective dose of a composition comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, until the skin disorder is cured, prevented or alleviated.

In some embodiments, there is provided a regimen of administration comprising the once daily or twice daily administration to a skin disorder subject in need thereof a therapeutically effective amount of a dosage form comprising a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram, cilomilast, ibudilast, piclamilast, pharmaceutically acceptable salt thereof and combinations thereof, optionally further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof and a carrier suitable for topical administration, wherein said composition consisting of two separate compositions, a first composition comprising tapinarof and at least one PDE4 inhibitor and a second composition comprising at least one additional active agent, selected from a retinoid, benzoyl peroxide (BPO), a Janus kinase inhibitor (JAK inhibitor), a corticosteroid of potency class 1-4, an acaricide and combinations thereof, wherein the at least one retinoid is selected from tretinoin, adapalene, tazarotene and combinations thereof, wherein the at least one JAK inhibitor is selected from tofacitinib, abrocitinib, delgocitinib, ruxolitinib and combinations thereof and wherein the at least one acaricide is selected from permethrin, ivermectin, crotamiton and combinations thereof and wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

In some embodiments, there is provided a kit comprising instructions for use and one or more dosage forms comprising a composition comprising from about 0.25% w/w to about 3.0% w/w tapinarof, from about 0.25% w/w to about 3.0% w/w at least one PDE4 inhibitor selected from roflumilast, roflumilast N-oxide, apremilast, crisaborole, rolipram. cilomilast, ibudilast, piclamilast, pharmaceutically acceptable salt thereof and combinations thereof, optionally further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof and a carrier suitable for topical administration, wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

In the examples below, all % values referring to a solution are in (w/w).

All % values, referring to dispersions (suspensions) are in (w/w).

Unless otherwise indicated, all solutions used in the example below refer to an aqueous solution of the indicated ingredient.

Tables 1-3 provide exemplary active agents' combination of active agent classes and specific active agents of this invention:

All the exemplary combinations of specific active agents comprise micronized tapinarof, micronized PDE4 inhibitor selected from roflumilast, N-oxide roflumilast, apremilast, crisaborole, rolipram, cilomilast, ibudilast, piclamilast and combinations thereof and optionally an additional active agent selected from a retinoid (selected from tretinoin, adapalene and tazarotene), benzoyl peroxide (BPO), a JAK inhibitor (selected from tofacitinib, abrocitinib, delgocitinib and ruxolitinib), a corticosteroid (selected from potency groups 1-4, e.g. halobetasol) an acaricide (selected from permethrin, ivermectin and crotamiton) and combinations thereof. The strength ranges of all the active agents in the exemplary compositions are detailed in Table 3, comprising 20 exemplary combination compositions.

Tapinarof in the exemplary compositions may be used as such, as detailed in Examples 8-10, or encapsulated as detailed in Examples 1-7. The other active agents in the combination compositions (an PDE4 inhibitor and optionally an additional active agent) may also be used as such, or encapsulated by a similar process, as detailed in Examples 3-7.

### EXAMPLES

### Example 1: Preparation of Silicone Dioxide (SiO₂) Encapsulated Tapinarof Dispersed in Water (15% w/w Tapinarof)

### Preparation of Tapinarof Dispersion and Acid Cocktail

Tapinarof dispersion is prepared by mixing 378 grams of CTAC CT-429 (Cetrimonium Chloride 30%), 6,756 grams of micronized tapinarof having an average particle size of less than 1 μm, and 18,855 grams water under high shear. The dispersion is homogenized for 60 min at 33° C. (no more than 45° C.).

An acid cocktail is prepared using 1013 grams Hydrochloric Acid (37%), 215.3 grams anhydrous Citric Acid, 322.3 grams Lactic Acid (90%), and 1632 grams water.

### Coating Cycle

The coating cycle is started by adding 953 grams sodium silicate solution extra pure (28%) to the tapinarof dispersion prepared in step a) under high shear, followed by adding the acid cocktail prepared in step (a) and followed by adding 1675 grams PDAC (3%) solution to the mixture. The cycle is repeated another 5 times. After the 6 cycles, the pH of the mixture is adjusted to 5.0 using the acid cocktail, and water is added to complete the total weight of the mixture to 45 kilograms.

The composition of the final tapinarof water suspension product is shown in Table 4.

TABLE 4
Composition of the encapsulated tapinarof 15% water suspension

| Ingredient | % w/w of ingredient in the suspension |
|---|---|
| Polyquarternium-7 | 5.6 |
| Hydrochloric Acid 37% | 2.0 |
| Citric Acid, Anhydrous | 0.4 |
| Lactic Acid | 0.6 |
| Silicone Dioxide | 3.4 |
| Sodium Hydroxide | 1.4 |
| Cetrimonium Chloride | 0.84 |
| Tapinarof | 15.00 |
| Sterile Water for Irrigation | Upto 100% |

### Example 2: Preparation of Encapsulated Roflumilast (15% E-Roflumilast Water Suspension)

### a) Preparation of Roflumilast Dispersion and Acid Cocktail

Roflumilast dispersion is prepared by mixing 125.67 grams of CTAC CT-429 (Cetrimonium Chloride 30%), 3008 grams of Roflumilast, and 5200 grams water under high shear. The solution is homogenized for 60 minutes at 33° C. (no more than 45° C.), and then the pH of the solution is adjusted to 7.0 using sodium hydroxide solution (20%).

An acid cocktail is prepared using 493 grams Hydrochloric acid (37%), 98 grams anhydrous Citric Acid, 147 grams Lactic Acid (90%), and 794 grams water.

### b) Coating Cycle

The coating cycle is started by adding 38 grams sodium silicate solution extra pure (28%) to the Roflumilast solution prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 4

TABLE 4
Composition of the encapsulated Roflumilast 15% water suspension

| Ingredient | % of ingredient in the suspension |
|---|---|
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

### Example 3: Preparation of Formulation of Encapsulated Roflumilast (1.5%) and Encapsulated Tapinarof (1.5%) in Emulsion (Formulation I)

Oil Phase: 720.0 of grams Cyclomethicone 5-N, 540.0 of grams Cetyl Alcohol, 360.0 grams Polyoxyl 100 Stearate, and 540.0 grams of Glyceryl Monosterate are mixed at 70° C.

Water phase: 18.0 grams of Ethylendiaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials are completely melted and dissolved. 76.5 grams if sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes. 1,800 grams of encapsulated Roflumilast 15% water suspension made as described in Example 2 are mixed. The resulting mixture is added to the emulsion at 65° C. and mixed at 1400 rpm for 10 minutes. The emulsion is cooled to 35° C. and the pH of the emulsion is adjusted to 3.5 using HCl 5N solution. After 1,800 grams of encapsulated Tapinarof 15% water suspension made as described in Example 1 are added, the emulsion is stirred at 1400 rpm for 10 minutes. HCl 5N is added to adjust the pH to 3.6, and then water was added until the total weight of the emulsion reached 18 kilograms. The composition of the formulation prepared in this example is shown in Table 5.

TABLE 5
Composition of Formulation I

| | % of pure ingredient in the |
|---|---|
| Ingredient | composition |
| Polyquarternium-7 | 0.11 |
| Hydrochloric Acid | 0.26 |
| Citric Acid, Anhydrous | 0.28 |
| Lactic Acid | 0.12 |
| Silicon Dioxide | 0.77 |
| Sodium hydroxide | 0.05 |
| Cetrimonium Chloride | 0.06 |
| Roflumilast | 1.50 |
| Tapinarof | 1.50 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | Up 100% |

### Example 4: Preparation of Tapinarof/Roflumilast Ointment Composition

The topical tapinarof/roflumilast ointment consists of:

0.25-3.0% w/w tapinarof,

0.25-3.0% w/w roflumilast

50-65% w/w white petrolatum,

6% woolfat,

15-30% w/w liquid paraffin,

3-10% w/w PEG-4000 or PEG-400

0.5% phenylethyl alcohol

0.1-1.0% w/w alpha-tocopherol

The ointment composition is prepared by the following steps:
1. Weigh liquid paraffin in a bottle. Remove air bubbles in the liquid paraffin by applying vacuum for a period of 30 minutes.
2. Melt white petrolatum and wool fat on a stir plate at approximately 70° C.
3. Mix gently the liquid paraffin, white petrolatum and wool fat at approximately 70° C. for a period of 15 minutes or until the ingredients are uniformly mixed.
4. Weigh tapinarof and roflumilast having an average particle size of less than 1 μm and dissolve them in PEG 400 or PEG 4000 by sonicating in a water bath maintained at 45° C.
5. Add alpha-tocopherol and phenylethyl alcohol to the PEG containing tapinarof/roflumilast.
6. Fill the tapinarof/roflumilast ointment in a tube or other delivery system.

### Example 5: Preparation of a Non-Encapsulated Tapinarof-Roflumilast Combination Composition

The topical tapinarof-roflumilast combination composition consists of:

0.25-3.0% w/w tapinarof,

0.25-3.0% w/w roflumilast

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

Adjust to pH 6.0-7.0 with 0.1M NaOH or 0.1M HCl

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, roflumilast, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-roflumilast combination cream in an aluminum tube or other delivery system.

### Example 6: Preparation of a Non-Encapsulated Tapinarof-Apremilast Combination Composition

The topical tapinarof-apremilast combination cream consists of:

0.25-3.0% w/w tapinarof,

0.25-3.0% w/w apremilast

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

Adjust to pH 6.0-7.0 with 0.1M NaOH or 0.1M HCl,

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, apremilast, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-apremilast combination cream in an aluminum tube or other delivery system.

### Example 7: Preparation of a Non-encapsulated Tapinarof-Roflumilast-Tretinoin Triple Combination Composition

The topical tapinarof-roflumilast-tretinoin triple combination cream consists of:

0.25-3.0% w/w tapinarof,

0.25-3.0% w/w roflumilast,

0.025%-0.5% w/w tretinoin,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

Adjust to pH 6.0-7.0 with 0.1M NaOH or 0.1M HCl

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, roflumilast, tretinoin, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-roflumilast-tretinoin combination cream in an aluminum tube or other delivery system.

### Examples 8-27: Preparation of Encapsulated Tapinarof-PDE4 Inhibitor and Optionally Additional Active Agent Combination Compositions

The exemplary compositions 1-20 of Table 3 are prepared in encapsulated form by using the proper process selected from Examples 1-3.

### Examples 28-47: Preparation of Non-Encapsulated Tapinarof-PDE4 Inhibitor and Optionally Additional Active Agent Combination Compositions

The exemplary compositions 1-20 of Table 3 are prepared in non-encapsulated form by using the proper process selected from Examples 4-7.

1. A topical composition comprising from about 0.25% w/w to about 3.0% w/w tapinarof, from about 0.25% w/w to about 3.0% w/w at least one PDE4 inhibitor and a carrier suitable for topical administration.

2. The composition of claim 1, wherein the at least one PDE4 inhibitor is selected from roflumilast, roflumilast N-oxide, apremilast, crisaborole, rolipram, cilomilast, ibudilast, piclamilast, pharmaceutically acceptable salt thereof and combinations thereof.

3. The composition of claim 1, wherein the PDE4 inhibitor is roflumilast, roflumilast N-oxide or pharmaceutically acceptable salt thereof.

4. The composition of claim 1, wherein the PDE4 inhibitor is apremilast.

5. The composition of claim 1, wherein the PDE4 inhibitor is crisaborole.

6. The composition of claim 1, further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof.

7. The composition of claim 6, wherein the composition consists of two separate compositions, a first composition comprising tapinarof and at least one PDE4 inhibitor and a second composition comprising at least one additional active agent, selected from a retinoid, benzoyl peroxide (BPO), a Janus kinase inhibitor (JAK inhibitor), a corticosteroid of potency class 1-4, an acaricide and combinations thereof.

8. The composition of claim 7, wherein the at least one retinoid is selected from tretinoin, adapalene, tazarotene and combinations thereof.

9. The composition of claim 7, wherein the at least one JAK inhibitor is selected from tofacitinib, abrocitinib, delgocitinib, ruxolitinib and combinations thereof.

10. The composition of claim 7, wherein the at least one acaricide is selected from permethrin, ivermectin, crotamiton and combinations thereof.

11. The composition of claim 6, wherein at least one of the active agents selected from tapinarof, at least one PDE4 inhibitor and the optional at least one additional active agent is encapsulated.

12. A dosage form comprising the composition of claim 1, wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

13. A method of treatment, prevention or alleviation of a skin disorder, by topical administration to the affected area of a skin disorder subject in need thereof of a therapeutically effective amount of a composition, comprising from about 0.25% w/w to about 3.0% w/w tapinarof, from about 0.25% w/w to about 3% w/w at least one PDE4 inhibitor and optionally at least one additional active agent selected from about 0.025% w/w to about 0.5% w/w of at least one retinoid; from about 2% w/w to about 10% w/w benzoyl peroxide (BPO); from about 0.1% w/w to about 3.0% w/w at least one Janus kinase inhibitor (JAK inhibitor); from about 0.01% w/w to about 0.25% w/w at least one corticosteroid of potency class 1-4, from about 0.5% to about 5% w/w; at least one acaricide; and combinations thereof.

14. The method of claim 13, wherein the topical administration is made to the affected skin area of a subject in need thereof as two separate compositions, a first composition comprising tapinarof and said at least one PDE4 inhibitor and a second composition comprising said at least one additional active agent, to be administered from two application syringes or from a dual chamber application syringe, simultaneously or sequentially in either order, wherein the two compositions are mixed on the skin of said subject in need thereof.

15. The method of claim 13, wherein the skin disorder is selected from psoriasis, dermatitis, acne, rosacea, ichthyosis, scaling skin, imbalance of skin barrier and tinea.

16. The method of claim 15, wherein the skin disorder is psoriasis, selected from plaque psoriasis, scalp psoriasis, flexural/inverse psoriasis, sebopsoriasis and genital psoriasis.

17. The method of claim 15, wherein the skin disorder is acne, selected from acne vulgaris, papulopustular acne and nodular acne.

18. The method of claim 15, wherein the skin disorder is rosacea, selected from erythematotelangiectatic rosacea, papulopustular rosacea, rhinophyma rosacea and ocular rosacea.

19. The method of claim 15, wherein the skin disorder is dermatitis (eczema), selected from atopic dermatitis, contact dermatitis, dyshidrotic dermatitis, nummular dermatitis and seborrheic dermatitis.

20. The method of claim 15, wherein the skin disorder is tinea, selected from tinea pedis, tinea capitis, tinea barbae, tinea faciei, tinea corporis, tinea manum, tinea cruris and tinea interdigital.

21. The method of claim 13, wherein the treatment comprises once daily or twice daily topical administration to a skin disorder subject in need thereof of a therapeutically effective amount of said composition.

22. The method of claim 13, wherein said tapinarof and the at least one PDE4 inhibitor and the optional at least one additional active agent exhibit an additive or synergistic effect, thereby allowing to reduce the amounts of the active agents in the composition.

23. The method of claim 13, wherein said at least one PDE4 inhibitor cancels or diminishes the tapinarof folliculitis side-effect.

24. A regimen of administration comprising the once daily or twice daily administration to a skin disorder subject in need thereof of a therapeutically effective dose of the composition of claim 1 until the skin disorder is cured, prevented or alleviated.

25. A regimen of administration comprising the once daily or twice daily administration to a skin disorder subject in need thereof a therapeutically effective amount of the dosage form of claim 12.

26. A kit comprising one or more dosage forms of claim 12 and instructions for use.

27. The method of claim 14, wherein the skin disorder is selected from psoriasis, dermatitis, acne, rosacea, ichthyosis, scaling skin, imbalance of skin barrier and tinea.

28. The composition of claim 1, wherein at least one of the active agents selected from tapinarof and at least one PDE4 inhibitor is encapsulated.
